SEQUENCE LISTING (3 sequences)

SEQ ID NO: 1 - protein, 182 aa, Gossypium spp.
MGSLDSKESPANNPGLHSPPDEATKGYIMQQTMFRIKDPKPTLEFYSRVLGMSLLNKVDVPYMKMTLYMMGYEDVSSAPSDPVEKTIWTFGRPATMELTHFWGTENDPEFKGYHDGNSEPTGFGHIGITVDDMYKACERFESLGVEFVKKPGDGYAFIKDPDGYWIEIFDLNGIRAIVNNLA

SEQ ID NO: 2 - protein, 182 aa, Gossypium spp.
MASLDSKESPANNPGLHFPPDEATKGYIMQQTMFRIKDPKRTLEFYSRVLGMTLLNKVDVPYMKMTLYMMGYEDVSSAPTDPVEKTIWTFGRPATMELTHFWGTENDPEFKGYHDGNSEPIGFGHIGLTVDDLYKACERFESLGVEFVKKPSDGFAFIKDPDGYWIEIFDLKGIRQIVNSLA

SEQ ID NO: 3 - protein, 182 aa, Gossypium ssp.
MGSMDSKESPANNPGLHTPPDEATKGYIMQQTMFRIKDPKRTLEFYSRVLGMTLLNKVDVPYMKMTLYMMGYEDVSSAPNDPVEKTIWTFGRPATMELTHFWGTENDPEFKGYHDGNSEPIGFGHIGITVDDMYKACERFESLGVEFVKKPSDGFAFIKDPDGYWIEIFDLKGIRQIVNSLA

(19) United States
(12) Patent Application Publication
(10) Pub. No.: US 2023/0399674 A1
(43) Pub. Date: Dec. 14, 2023

(54) METHODS AND COMPOSITIONS FOR DEGRADING DEOXYNIVALENOL

(71) Applicant: ERBER AKTIENGESELLSCHAFT, Getzersdorf (AT)

(72) Inventors: Wulf-Dieter Moll, Stockerau (AT); Barbara Weber, Wien (AT); Martin Siller, Wien (AT)

(21) Appl. No.: 18/032,312

(22) PCT Filed: Oct. 22, 2021
(86) PCT No.: PCT/EP2021/079331

§ 371 Date: Apr. 17, 2023

(30) Foreign Application Priority Data
Oct. 22, 2020 (EP) 20203359.3

(51) Int. Cl. C12P 41/00; A23K 20/147; A23K 20/189; A23L 5/20; A23L 33/18; C12N 9/04

(52) U.S. Cl. CPC C12P 41/00; A23K 20/147; A23K 20/189; A23L 5/25; A23L 33/18; C12N 9/0006; C12Y 101/01001

(57) ABSTRACT

The present invention relates to means and methods for degrading DON and/or DON derivative/s comprising a polypeptide comprising an amino acid sequence having at least 70% identity to the amino acid sequence set forth in SEQ ID NO: 1.

This application contains a Sequence Listing in a computer readable form, which is incorporated herein by reference.

### TECHNICAL FIELD

The present invention relates to means and methods for degrading DON and/or DON derivative/s, said means and methods comprising a polypeptide having at least 70% sequence identity to the amino acid sequence set forth in SEQ ID NO: 1.

### BACKGROUND OF THE INVENTION

Mycotoxins are secondary metabolites of filamentous fungi. These fungi grow, inter alia, on various types of grain and cereals. Typically, the crops are infested by the fungi prior to harvest and mycotoxin production can occur before and after harvest as well as during storage. According to the Food and Agriculture Organization, approximately 25% of all agricultural products are contaminated with mycotoxins (e.g., Eskola et al. (2020), Crit. Rev. Food Sci. Nutr. 60(16), 2773-2789) causing considerable economic losses. Among 19,757 samples analyzed from January 2004 to December 2011, 72% of the samples tested positive for at least one mycotoxin, and 39% were co-contaminated (Schatzmayr and Streit. 2013. World Mycotoxin Journal 6(3): 213-222). Mycotoxins are known to cause serious adverse health effects in humans and animals, including mutagenic, cancerogenic, neurotoxic, immunosuppressive or teratogenic effects. A particularly large group of mycotoxins are trichothecene mycotoxins or trichothecenes. Trichothecenes are a class of sesquiterpenes which are produced inter alia by Fusarium, Myrothecium, Podostroma, Trichoderma or Trichothecium species and may comprise an 12,13-epoxy ring (that may interchangeably be referred to herein as “12,13-epoxytrichothec-9-ene moiety” or “trichothecene-ring”) as a common structural feature.

The family of trichothecene mycotoxins comprises, among several others, deoxynivalenol (DON, CAS no. 51481-10-8), T-2 toxin (CAS no. 21259-20-1), HT-2 toxin (CAS no. 26934-87-2), nivalenol (CAS no. 23282-20-4), fuseranon-X (CAS no. 23255-69-8), scripentriol, 15-acetoxyscirpenol (CAS no. 2623-22-5), 4,15-diacetoxyscirpenol (CAS no. 2270-40-8), trichodermol (CAS no. 2198-93-8), verrucarin A (CAS no. 3148-09-2), verrucarin J (CAS no. 4643-58-7), isotrichodermin (CAS no. 91423-90-4), hydroxyisotrichodermin (CAS no. 344781-02-8), calonectrin (CAS no. 38818-51-8), T-2 tetraol (CAS no. 34114-99-3), deacetylneosolaniol (CAS no. 74833-39-9), neosolaniol (CAS no. 36519-25-2), acetylneosolaniol (CAS no. 65041-92-1), sporotrichiol (CAS no. 101401-89-2) and trichotriol (CAS no. 109890-37-1).

Type B trichothecenes as referred herein may refer to trichothecene compounds having a carbonyl group (e.g., C═O) at the position 8 of the carbon chain (e.g., see below), according to the atom numbering by Yoshizawa and Morooka (1973, Agric. Biol. Chem. 37, 2933-2934), (or, in other words, an oxygen atom (e.g., ═O), doubly bonded to the C8-atom of the carbon chain) and may include DON-derivatives. DON-derivatives as referred herein may refer to trichothecene compounds having a carbonyl group (e.g., C═O) at the position 8 of the carbon chain (or, in other words, an oxygen atom (═O), doubly bonded to the C8-atom of the carbon chain) and a hydroxyl group (—OH) at the C7-atom of the carbon chain and an unsubstituted C10 (e.g., having hydrogen (—H) bound to it, e.g., see DON chemical structure below). Exemplary DON-derivatives as referred herein may include:

| Name | Abbreviation | Chemical | Formula | MW | CAS no |
|---|---|---|---|---|---|
| Deoxynivalenol | DON | (3α, 7α)-3,7,15-trihydroxy- | C₁₅H₂₀O₆ | 296.3 | 51481-10- |
| | | 12,13-epoxytrichothec-9-en- | | | 8 |
| | | 8-one | | | |
| 3-keto | 3K-DON | (7α)-7,15-dihydroxy-12,13- | C₁₅H₁₈O₆ | 294.3 | Not |
| Deoxynivalenol | | epoxytrichothec-9-en-3,8-one | | | available |
| 3-epi | 3E-DON | (3β, 7α)-3,7,15-trihydroxy- | C₁₅H₂₀O₆ | 296.3 | Not |
| Deoxynivalenol | | 12,13-epoxytrichothec-9-en- | | | available |
| | | 8-one | | | |
| 15- | 15A-DON | 15-Acetyloxy-3α,7α- | C₁₇H₂₂O₇ | 338.4 | 88337-96- |
| Acetyldeoxynivalenol | | dihydroxy-12,13- | | | 6 |
| | | epoxytrichothec-9-en-8-one | | | |
| Nivalenol | NIV | (3α,4β,7α)-3,4,7,15- | C₁₅H₂₀O₇ | 312.3 | 23282-20- |
| | | tetrahydroxy-12,13- | | | 4 |
| | | epoxytrichothec-9-en-8-one | | | |
| Fusarenon-X (4- | FusX | (3β, 4α,7α)-3,7,15-trihydroxy- | C₁₇H₂₂O₈ | 354.4 | 23255-69- |
| acetylnivalenol) | | 8-oxo-12,13-epoxytrichothec- | | | 8 |
| | | 9-en-4-yl acetate | | | |
| 3-amino | 3-amino | 3-amino-(7α)-7,15- | C₁₅H₂₁NO₅ | 295.3 | Not |
| Deoxynivalenol | DON | dihydroxy-12,13- | | | available |
| | | epoxytrichothec-9-en-8-one | | | |

DON-derivatives as referred herein do not include 3-Ac-DON, i.e., 3-Acetyloxy-3α, 7α-dihydroxy-12,13-epoxytrichothec-9-en-8-one.

In animals, the consumption of trichothecene mycotoxins leads to reduced feed intake, impaired nutrient absorption and reduced growth, vomiting, diarrhea, immunological dysfunctions and reduced milk production. In humans, adverse health effects include nausea, vomiting, diarrhea, abdominal pains, headaches and fever. Especially crop contamination with DON (chemical name e.g., (3α,7α)-3,7,15-trihydroxy-12,13-epoxytrichothec-9-en-8-one) can be found all around the world and several additional toxic DON derivatives have been described, e.g. acetylated DON (such as 15-acetyl-DON, 15-ADON), glycosylated DON, DON sulfonate (such as DONS-1 or DONS-2), or DON sulfate (such as DON-15-sulfate). National and regional authorities have therefore established maximum levels for DON in food (EC no. 1881/2006, EC no. 1126/2007) or feed (2006/576/EC) to avoid mycotoxicosis of humans and animals upon DON ingestion, leading to undesirable effects such as the inhibition of protein biosynthesis, interactions with serotonin and dopamine receptors or upregulation of pro-inflammatory cytokines (EFSA Journal 2004, 73, 1-41).

The risk of contamination with some mycotoxins can be reduced by applying “good agricultural practice”. Despite such precautions, DON contamination cannot be avoided and additional strategies are necessary to avoid DON mycotoxicosis. A possible strategy to combat mycotoxicosis is the addition of binding agents such as clays, yeast or yeast products to the feed and/or food. However, due to its hydrophilic character, DON does not interact with commonly employed binding agents and thus cannot be removed by such a strategy. Similarly, DON cannot be removed from food or feed by chemical or physical treatment. As an alternative strategy to avoid mycotoxicosis, DON could be detoxified by transformation to a less toxic molecule.

EP 1 042 449 A1 or US 2012/0263827 A1 relate to microorganisms capable of detoxifying trichothecenes by cleaving of the epoxy ring on the C12 and C13 atoms or by biotransformation to 3-epi-DON and 3-keto-DON, respectively. DON detoxification strategies using microorganisms based on the C3 atom are known in the art. Generally, the use of such microorganisms is often challenged by the specific cultivation requirements of these microorganisms, rendering their production economically unfavorable. In addition, the admixture of microorganisms to feed or food is not feasible in many food or feed processes. Therefore, biotransformation of DON by a microorganism-free enzyme formulation is needed as fast and safe means for DON detoxification. In this regard, WO 2016/154640 A1 describes an alcohol dehydrogenase containing metal ions and a quinone cofactor for transforming a trichothecene having a hydroxyl-group at the C3 atom. CN 107916266 A describes a multi-enzyme process for the biotransformation of DON to 3-epi-DON. In 2012, a three-enzyme system comprising the cytochrome P450 enzyme DdnA derived from Sphingomonas sp. KSM1 was described to perform a hydroxylation of DON to 16-hydroxy-DON (Ito et al. 2012. Appl Environ Microbiol 79(5):1619-1628).

The present invention was made in view of the prior art outlined above. The objective of the present invention can be inter alia formulated as providing novel means and methods to detoxify deoxynivalenol (DON). The solution of the present invention is described in the following, exemplified in the examples, illustrated in the figures and reflected in the claims.

This objective has been achieved by providing SEQ ID NO: 1 capable of modifying (e.g., detoxifying and/or degrading) DON to 7-one-8-hydroxy-8-ene-DON (e.g., FIG. 2, also known as 3,8,15-trihydroxy-12,13-epoxytrichothec-8-en-7-one). DON is a trichothecene mycotoxin having a carbonyl group (C═O) at the position 8 of the carbon chain (or, in other words, an oxygen atom (═O), doubly bonded to the C8-atom of the carbon chain) and a hydroxyl group (—OH) at the C7-atom of the carbon chain with the following structural formula:

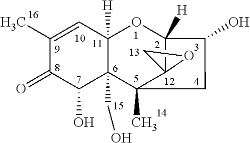 [SMILES: CC1=C[C@H]2O[C@@H]3[C@H](O)C[C@@](C)(C34CO4)[C@@]2(CO)[C@H](O)C1=O]

### SUMMARY OF THE INVENTION

The present invention relates to methods and compositions for degrading DON and/or DON derivative/s and/or altering toxicity of DON and/or DON derivative/s, said methods/compositions comprising: (i) providing one or more of the following polypeptides (preferably having not 100% identity to SEQ ID NOs: 2-3) selected from the group consisting of: (a) a polypeptide comprising the amino acid sequence set forth in SEQ ID NO: 1; (b) a polypeptide comprising an amino acid sequence having at least 70% (e.g., at least 80%, at least 85%, at least 90%, at least 91%, at least 92%, at least 93%, at least 94%, at least 95%, at least 96%, at least 97%, at least 98%, at least 99%) identity to the amino acid sequence set forth in SEQ ID NO: 1; (c) a variant of the polypeptide set forth in SEQ ID NO: 1, wherein said variant comprising: a substitution, deletion, and/or insertion at one or more positions; and/or (d) a fragment of the polypeptide of (a), (b) or (c), wherein said fragment is capable of degrading DON and/or DON derivatives and/or altering toxicity of DON and/or DON derivatives, preferably said altering toxicity comprising one or more of the following: detoxifying and/or changing intermolecular rearrangement of DON and/or DON derivatives; (ii) optionally, applying one or more of (a)-(d) to DON and/or DON derivative/s; preferably said one or more polypeptides are one or more recombinant and/or isolated polypeptides; further preferably said altering toxicity comprising one or more of the following: detoxifying and/or intramolecularly rearranging DON and/or DON derivative/s; most preferably said one or more polypeptides are capable of modifying (e.g., modifying bond/s and/or binding partner/s and/or functional group/s at C7 and/or causing a chemical change, e.g., breaking bonds and/or making new bonds, etc.) the C7-atom of DON (e.g., producing 7-one-8-hydroxy-8-ene-DON from DON) and/or DON derivatives.

The present application satisfies this demand by the provision of the means and method for degrading DON and/or DON derivatives described herein below, characterized in the claims and illustrated by the appended Examples and Figures.

### Overview of the Sequence Listing

SEQ ID NO: 1 is the amino acid sequence, which is suitable for modifying bonds at the C7-atom of DON (e.g., producing 7-one-8-hydroxy-8-ene-DON from DON).

SEQ ID NO: 2 is the amino acid sequence, which is not suitable for modifying bonds at the C7-atom of DON.

SEQ ID NO: 3 is the amino acid sequence, which is not suitable for modifying bonds at the C7-atom of DON.

### BRIEF DESCRIPTION OF THE DRAWINGS

FIG. 1: SDS-PAGE gel (12% Mini-PROTEAN (Bio-Rad) TGX stain-free precast gels) with fractions of lysate corresponding to 10 μl E. coli gene expression culture (T: total lysate; S: soluble fraction; I: insoluble fraction). SEQ ID NO: 1: 20.5 kDa. The 35.2 kDa reference protein was included as gene expression control.

FIG. 2: Molecular structures of DON and 7-one-8-hydroxy-8-ene-DON.

FIG. 3: Concentrations of DON and 7-one-8-hydroxy-8-ene-DON after incubation of crude E. coli cell lysates in reaction buffer with 100 μM DON for 3 h at 30° C.

FIG. 4: Time course of DON and 7-one-8-hydroxy-8-ene-DON concentrations in reaction buffer with initially nominally 100 μM DON, incubated with cleared lysates of E. coli BL21 (DE3) biomass from gene expression cultures.

### DETAILED DESCRIPTION OF THE INVENTION

### Definitions

As referred herein “EC numbers” (Enzyme Commission numbers) may be used to refer to enzymatic activity according to the Enzyme nomenclature database, Release of February 26, 2020 (e.g., available at https://enzyme.expasy.org/). The EC number refers to Enzyme Nomenclature 1992 from NC-IUBMB, Academic Press, San Diego, Calif., including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively.

The term “polypeptide” is equally used herein with the term “protein”. Proteins (including fragments thereof, preferably biologically active fragments, and peptides, usually having less than 30 amino acids) comprise one or more amino acids coupled to each other via a covalent peptide bond (resulting in a chain of amino acids). The term “polypeptide(s)” as used herein describes a group of molecules, which, for example, consist of more than 30 amino acids. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “polypeptide” and “protein” also refer to naturally modified polypeptides/proteins wherein the modification is affected e.g. by post-translational modifications like glycosylation, acetylation, phosphorylation and the like. Such modifications are well known in the art.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used may be gap open penalty of 10, gap extension penalty of 0.5, and the EBLOSUM62 (EMBOSS version of BLOSUM62) substitution matrix. The output of Needle labeled “longest identity” (obtained using the no-brief option) is used as the percent identity and is calculated as follows: (Identical Residues×100)/(Length of Alignment−Total Number of Gaps in Alignment).

Alternatively, the parameters used may be gap open penalty of 10, gap extension penalty of 0.5, and the EDNAFULL (EMBOSS version of NCBI NUC4.4) substitution matrix. The output of Needle labeled “longest identity” (obtained using the no-brief option) is used as the percent identity and is calculated as follows:

(Identical Deoxyribonucleotides×100)/(Length of Alignment−Total Number of Gaps in Alignment).

Expression: The term “expression” includes any step involved in the production of a variant (polypeptide) including, but not limited to, transcription, post-transcriptional modification, translation, post-translational modification, and secretion.

Expression vector: The term “expression vector” may refer to a linear or circular DNA molecule that comprises a polynucleotide encoding a variant (polypeptide) and is operably linked to control sequences that provide for its expression, in particular for its transcription.

Fragment: The term “fragment” may refer to a polypeptide having one or more (e.g. several, e.g., 5, 10, 20, 30, 40, etc.) amino acids absent from the amino and/or carboxyl terminus of a mature polypeptide; wherein the fragment has an activity as described elsewhere herein.

Host cell: The term “host cell” may refer to any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Nucleic acid construct: The term “nucleic acid construct” may refer to a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” may refer to a configuration in which a control sequence is placed at an appropriate position relative to the coding sequence of a polynucleotide such that the control sequence directs expression of the coding sequence.

Control sequences: The term “control sequences” as used herein may refer to nucleic acid sequences necessary for expression of a polynucleotide encoding a variant (polynucleotide) of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, pro-peptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide of the present invention.

As used herein, the term “corresponding to” may refer to a way of determining the specific amino acid of a sequence wherein reference is made to a specific amino acid sequence (e.g., US2020071638). E.g. for the purposes of the present invention, when references are made to specific amino acid positions, the skilled person would be able to align another amino acid sequence to said amino acid sequence that reference has been made to, in order to determine which specific amino acid may be of interest in said other amino acid sequence. Alignment of another amino acid sequence with e.g. the sequence as set forth in SEQ ID NOs: 1, 2, 3 or any other sequence listed herein, has been described elsewhere herein. Alternative alignment methods may be used, and are well-known for the skilled person.

The term “position” when used in accordance with the present invention may refer to a position of an amino acid within an amino acid sequence depicted herein. The term “corresponding” in this context may include that a position is not only determined by the number of the preceding nucleotides/amino acids.

As used herein, “silent” mutations mean base substitutions within a nucleic acid sequence which do not change the amino acid sequence encoded by the nucleic acid sequence. “Conservative or equivalent” substitutions (or mutations) mean substitutions as listed as “Exemplary Substitutions” in Table I below. “Highly conservative” substitutions as used herein mean substitutions as shown under the heading “Preferred Substitutions” in Table I below.

TABLE I
Amino Acid Substitutions

| | | Preferred |
|---|---|---|
| Original | Exemplary Substitutions | Substitutions |
| Ala (A) | val; leu; ile | Val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; | leu |
| Leu (L) | norleucine; ile; val; met; ala; | ile |
| Lys (K) | arg; gin; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | Phe |
| Val (V) | ile; leu; met; phe; ala; | leu |

Variant: The term “variant” may refer to a polypeptide having specific activity as described herein comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

As used herein the term “transgenic” may refer to an organism whose genome has been altered by the incorporation of foreign genetic material or additional copies of native genetic material, e.g. by transformation or recombination (e.g., U.S. Pat. No. 7,410,800B2). The transgenic organism may be a plant, mammal, fungus, bacterium or virus. As used herein “transgenic plant, seed or pollen grain” may refer to a plant, seed or pollen grain or progeny plant, seed or pollen grain of any subsequent generation derived therefrom, wherein the DNA of the plant, seed or pollen grain or progeny thereof contains an introduced exogenous DNA not originally present in a non-transgenic plant, seed or pollen grain of the same strain. The transgenic plant, seed or pollen grain may additionally contain sequences which are native to the plant being transformed, but wherein the exogenous DNA has been altered in order to alter the level or pattern of expression of the coding sequence.

The term “modifying the C7-atom” may refer to a process of intramolecular rearrangement and/or change of bond/s and/or binding partner/s and/or functional group/s at C7 atom of the carbon chain (e.g., position 7 of the carbon chain), e.g., of DON and/or DON derivative/s, (e.g., comprising changing (e.g., oxidizing) a hydroxyl-moiety (e.g., —OH) to carbonyl moiety (e.g., ═O) at the C7-atom (e.g., position 7 of the carbon chain) and/or causing a chemical change, e.g., breaking bond/s and/or making new bond/s.

The term “foodstuff” may refer to a substance having a food value.

The term “fodder” may refer to a substance fed to domestic animals.

The term “feed” may refer to a substance used as food for livestock.

The term “additive” may refer to a compound or substance added to another product or substance, e.g., for a technological purpose in the manufacture, processing, preparation, treatment, packaging, transport or storage of a foodstuff-, fodder- or feed product, e.g., in a small amount, to affect a desired property and/or characteristics. Exemplary additives of the present invention may include: processing aids (i.e., substance used in the production of food, but are not consumed as a food), reactants (i.e., substances that are consumed in the course of a chemical reaction) and catalysts (substances that increase the rate of a reaction without modifying the overall standard Gibbs energy change in the reaction) e.g., used in food or feed industry, starch production, e.g., citric acid, bioethanol, etc.

The term “prebiotic” may refer to a compound or substance capable of inducing the growth and/or activity of microorganisms (e.g., beneficial microorganisms).

The term “detoxifying agent” may refer to a compound or substance capable of reducing- and/or inhibiting toxicity of a e.g., mycotoxin, e.g., DON and/or DON derivative/s.

The term “nutritional supplement” may refer to a compound or substance capable of supporting the nutritional content of the diet, e.g., vitamins and minerals.

The term “intermediate” may refer to a compound or substance produced during the process (e.g., during an intermediate stage of the process) of obtaining an end-product of the present invention, e.g., foodstuff, fodder, fodder; feed, additive (e.g., foodstuff-, fodder- or feed additive), detoxifying agent, nutritional supplement or prebiotic of the present invention

The term “nutritive source” may refer to any substance that can be used for/in nutrition and/or biological energy production (e.g., comprising a carbohydrate and/or protein and/or fat).

The term “material” may refer to a raw material that is of plant origin, for example a citric acid, or bioethanol vegetable tuber or root, such as but not limited to the group consisting of potato, carrot, beet, parsnip, parsley root, celery root, sweet potato, yams, yam bean, radish, turnip, chicory root and cassava; cereal, such as but not limited to the group consisting of wheat, rice, corn, maize, rye, barley, buckwheat, sorghum, oats and ragi; coffee; cocoa; chicory; olives; prunes or raisins.

The term “intramolecularly rearranging” or “intermolecular rearrangement” may refer to any process that involves a transfer (e.g., of atoms, groups, electrons, bond/s, etc.) or interactions between different parts of the same molecular entity or between two or more molecular entities.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

In the course of the present invention it was found that the polypeptide SEQ ID NO: 1 was able to convert DON to 7-one-8-hydroxy-8-ene-DON (quantification is shown in FIG. 3), whereas the related SEQ ID NO: 2 and SEQ ID NO: 3 did not show any conversion of DON or any reduction of DON concentration. It is well known in the art, that 7-one-8-hydroxy-8-ene-DON is less toxic than DON (e.g., Stadler et al. (2019), Food Chem. 279, 303-311; Stadler et al. (2019), Toxins (Basel) 11, 317). Thus, it was surprisingly found that SEQ ID NO: 1 is able to detoxify DON. Accordingly, the objective of the present invention has been achieved by providing means, methods and products based on SEQ ID NO: 1 as described herein below.

In some embodiments, the present invention provides a method for producing a foodstuff, intermediate foodstuff; fodder, intermediate fodder; feed, intermediate feed; additive (e.g., foodstuff-, fodder- or feed additive, processing aid, reactant or catalyst, e.g., in food or feed industry, citric acid, bioethanol, starch production etc.), intermediate additive (e.g., foodstuff-, fodder- or feed intermediate additive, intermediate processing aid, reactant or catalyst, e.g., in food or feed industry, citric acid, bioethanol, starch production etc.); detoxifying agent, intermediate detoxifying agent; nutritional supplement, intermediate nutritional supplement; prebiotic, intermediate prebiotic and/or mixture/s thereof, said method comprising: (i) providing one or more of the following polypeptides: (a) a polypeptide comprising the amino acid sequence set forth in SEQ ID NO: 1; (b) a polypeptide comprising an amino acid sequence having at least 70% (e.g., at least 80%, at least 85%, at least 90%, at least 91%, at least 92%, at least 93%, at least 94%, at least 95%, at least 96%, at least 97%, at least 98%, at least 99%) identity to the amino acid sequence set forth in SEQ ID NO: 1; preferably said polypeptide does not have a 100% identity amino acid sequence set forth in SEQ ID NOs: 2 or 3; (c) a variant of the polypeptide set forth in SEQ ID NO: 1, wherein said variant comprising: a substitution, deletion, and/or insertion at one or more positions; preferably said variant does not have a 100% identity to amino acid sequence set forth in SEQ ID NOs: 2 or 3; and/or (d) a fragment of the polypeptide of (a), (b) or (c), wherein said fragment is capable of degrading DON (e.g., to 7-one-8-hydroxy-8-ene-DON) and/or DON derivatives (e.g., DON derivative may comprise a hydroxyl moiety (—OH) at the C7-atom, a carbonyl moiety (C═O) at position 8 of the carbon chain; C10-atom not substituted) and/or altering toxicity of DON (e.g., by producing 7-one-8-hydroxy-8-ene-DON) and/or DON derivative/s (e.g., DON derivative may comprise a hydroxyl moiety (—OH) at the C7-atom, a carbonyl moiety (═O) at C8; C10 not substituted), preferably said altering toxicity comprising one or more of the following: detoxifying and/or changing intermolecular rearrangement of DON (e.g., by producing 7-one-8-hydroxy-8-ene-DON) and/or DON derivatives; (ii) applying one or more of (a)-(d) to a nutritive source (e.g., comprising a carbohydrate and/or protein source) or material (e.g., raw material, crop, grain, citric acid, bioethanol, etc.) suitable for production of foodstuff, intermediate foodstuff; fodder, intermediate fodder; feed, intermediate feed; additive (e.g., foodstuff-, fodder- or feed additive, processing aid, reactant or catalyst, e.g., in food or feed industry, citric acid, bioethanol, starch production etc.), intermediate additive (e.g., foodstuff-, fodder- or feed intermediate additive, intermediate processing aid, reactant or catalyst, e.g., in food or feed industry, citric acid, bioethanol, starch production etc.); detoxifying agent, intermediate detoxifying agent; nutritional supplement, intermediate nutritional supplement; prebiotic, intermediate prebiotic and/or mixture/s thereof; preferably said one or more polypeptides are one or more recombinant and/or isolated polypeptides; further preferably said one or more polypeptides are capable of modifying the C7-atom of DON (e.g., by producing 7-one-8-hydroxy-8-ene-DON) and/or DON derivative/s; most preferably said modifying comprising changing a hydroxyl-moiety (e.g., can be referred to as —OH or C—OH) to carbonyl moiety (e.g., can be referred to as ═O or C═O) at the C7-atom (e.g., position 7); further most preferably said DON derivative is not 3-Ac-DON, i.e., 3-Acetyloxy-3α,7α-dihydroxy-12,13-epoxytrichothec-9-en-8-one).

In other embodiments, the method of the present invention further comprising: incubating the nutritive source or material with one or more of (a)-(d) under conditions suitable for degrading DON and/or DON derivative/s and/or altering toxicity of DON and/or DON derivative/s; preferably said method further comprising heat-treating and/or fractionating and/or drying the product of said incubation; further preferably said altering toxicity comprising one or more of the following: detoxifying (e.g., by producing 7-one-8-hydroxy-8-ene-DON) and/or intramolecularly rearranging DON (e.g., by producing 7-one-8-hydroxy-8-ene-DON) and/or DON derivative/s (preferably said DON derivative is not 3-Ac-DON, i.e., 3-Acetyloxy-3α,7α-dihydroxy-12,13-epoxytrichothec-9-en-8-one).

In other embodiments, the present invention relates to a foodstuff, intermediate foodstuff; fodder, intermediate fodder; feed, intermediate feed; additive (e.g., foodstuff-, fodder- or feed additive, processing aid, reactant or catalyst, e.g., in food or feed industry, citric acid, bioethanol, starch production etc.), intermediate additive (e.g., foodstuff-, fodder- or feed intermediate additive, intermediate processing aid, reactant or catalyst, e.g., in food or feed industry, citric acid, bioethanol, starch production etc.); detoxifying agent, intermediate detoxifying agent; nutritional supplement, intermediate nutritional supplement; prebiotic, intermediate prebiotic and/or mixture/s thereof produced by the method of the present invention.

In some embodiments, the present invention provides a method for degrading DON (e.g., by producing 7-one-8-hydroxy-8-ene-DON) and/or DON derivative/s and/or altering toxicity of DON (e.g., by producing 7-one-8-hydroxy-8-ene-DON) and/or DON derivative/s (e.g., DON derivative may comprise a hydroxyl moiety (—OH) at the C7-atom, a carbonyl moiety (C═O) at position 8 of the carbon chain; C10 not substituted; said degrading and/or altering toxicity comprising: modifying the C7-atom of DON and/or DON derivative/s; preferably said DON derivative is not 3-Ac-DON, i.e., 3-Acetyloxy-3α,7α-dihydroxy-12,13-epoxytrichothec-9-en-8-one), said method comprising: (i) providing one or more of the following polypeptides: (a) a polypeptide comprising the amino acid sequence set forth in SEQ ID NO: 1; (b) a polypeptide comprising an amino acid sequence having at least 70% (e.g., at least 80%, at least 85%, at least 90%, at least 91%, at least 92%, at least 93%, at least 94%, at least 95%, at least 96%, at least 97%, at least 98%, at least 99%) identity to the amino acid sequence set forth in SEQ ID NO: 1, preferably said polypeptide is not having 100% identity to SEQ ID NO: 2-3; (c) a variant of the polypeptide set forth in SEQ ID NO: 1, wherein said variant comprising: a substitution, deletion, and/or insertion at one or more positions; preferably said variant is not having 100% identity to SEQ ID NO: 2-3; and/or (d) a fragment of the polypeptide of (a), (b) or (c), wherein said fragment is capable of degrading DON (e.g., by producing 7-one-8-hydroxy-8-ene-DON) and/or DON derivative/s and/or altering toxicity of DON (e.g., by producing 7-one-8-hydroxy-8-ene-DON) and/or DON derivative/s, preferably said altering toxicity comprising one or more of the following: detoxifying and/or changing intermolecular rearrangement of DON (e.g., by producing 7-one-8-hydroxy-8-ene-DON) and/or DON derivative/s; (ii) applying one or more of (a)-(d) to DON and/or DON derivative/s; preferably said one or more polypeptides are one or more recombinant and/or isolated polypeptides; further preferably said altering toxicity comprising one or more of the following: detoxifying, and/or intramolecularly rearranging DON (e.g., by producing 7-one-8-hydroxy-8-ene-DON) and/or DON derivative/s (preferably said DON derivative is not 3-Ac-DON, i.e., 3-Acetyloxy-3α,7α-dihydroxy-12,13-epoxytrichothec-9-en-8-one)); most preferably said one or more polypeptides are capable of modifying the C7-atom of DON and/or DON derivatives (e.g., said modifying comprising changing C—OH to C═O moiety, e.g., at position 7; preferably said DON derivative is not 3-Ac-DON, i.e., 3-Acetyloxy-3α,7α-dihydroxy-12,13-epoxytrichothec-9-en-8-one).

In some embodiments, the present invention provides a composition or kit, comprising: (i) a polypeptide capable of degrading DON (e.g., by producing 7-one-8-hydroxy-8-ene-DON) and/or DON derivative/s (e.g., DON derivative may comprise a hydroxyl moiety (—OH) at the C7-atom, a carbonyl moiety (e.g., C═O) at position 8 of the carbon chain; C10 not substituted; preferably said DON derivative is not 3-Ac-DON, i.e., 3-Acetyloxy-3α,7α-dihydroxy-12,13-epoxytrichothec-9-en-8-one) and/or altering toxicity of DON (e.g., by producing 7-one-8-hydroxy-8-ene-DON) and/or DON derivative/s, wherein said polypeptide is one or more of the following: (a) a polypeptide comprising the amino acid sequence set forth in SEQ ID NO: 1; (b) a polypeptide comprising an amino acid sequence having at least 70% (e.g., at least 80%, at least 85%, at least 90%, at least 91%, at least 92%, at least 93%, at least 94%, at least 95%, at least 96%, at least 97%, at least 98%, at least 99%) identity to the amino acid sequence set forth in SEQ ID NO: 1; preferably said polypeptide is not having 100% identity to SEQ ID NO: 2-3; (c) a variant of the polypeptide set forth in SEQ ID NO: 1, wherein said variant comprising: a substitution, deletion, and/or insertion at one or more positions; preferably said variant is not having 100% identity to SEQ ID NO: 2-3;and/or (d) a fragment of the polypeptide of (a), (b) or (c), wherein said fragment is capable of degrading DON (e.g., by producing 7-one-8-hydroxy-8-ene-DON) and/or DON derivatives and/or altering toxicity of DON (e.g., by producing 7-one-8-hydroxy-8-ene-DON) and/or DON derivatives, preferably said altering toxicity comprising one or more of the following: detoxifying and/or changing intermolecular rearrangement of DON (e.g., by producing 7-one-8-hydroxy-8-ene-DON) and/or DON derivative/s (preferably said DON derivative is not 3-Ac-DON, i.e., 3-Acetyloxy-3α,7α-dihydroxy-12,13-epoxytrichothec-9-en-8-one); (ii) a nutritive source (e.g., comprising one or more of the following: carbohydrate, protein, citric acid, ethanol, e.g., bioethanol; preferably said nutritive source is not 3-Ac-DON) and/or a substrate for at least one of the polypeptides (a)-(d), preferably said substrate comprising: DON and/or DON derivative/s (preferably, said substrate is not 3-Ac-DON; further preferably said composition further comprising the product of the reaction (e.g., enzymatic reaction) of said one or more polypeptides with DON (e.g., 7-one-8-hydroxy-8-ene-DON) and/or DON derivative/s, i.e., a reaction product comprising a modification at the C7-atom of DON and/or DON derivative/s); preferably said polypeptide is a recombinant and/or isolated polypeptide; further preferably said altering toxicity comprising one or more of the following: detoxifying, and/or intramolecularly rearranging DON and/or DON derivative/s (preferably, DON derivative is not 3-Ac-DON); most preferably said polypeptide is capable of modifying the C7-atom of DON and/or DON derivatives (preferably, said modifying comprising changing C—OH to C═O moiety, e.g., at position 7; further preferably said DON derivative is not 3-Ac-DON).

In other embodiments, said one or more polypeptides of the present invention are encoded by one or more nucleotide sequences.

In other embodiments, said one or more nucleotide sequences of the present invention are comprised by one or more nucleic acids comprised by a host cell.

In other embodiments, the composition or kit of the present invention is one or more of the following: cell-free and/or non-naturally occurring and/or fractionated composition or kit.

In other embodiments, the composition or kit of the present invention is a pharmaceutical or veterinary composition or kit.

In other embodiments, the composition, kit, foodstuff, intermediate foodstuff; fodder, intermediate fodder; feed, intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement; prebiotic, intermediate prebiotic or mixture/s thereof of the present invention can be used as a medicament (e.g., including veterinary use) and/or in therapy.

In other embodiments, the composition, kit, foodstuff, intermediate foodstuff; fodder, intermediate fodder; feed, intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement; prebiotic, intermediate prebiotic or mixture/s thereof of the present invention can be used in treatment, amelioration, prophylaxis and/or diagnostics of DON mycotoxicosis.

In some embodiments, the present invention provides a method for modifying the C7-atom of DON (e.g., by producing 7-one-8-hydroxy-8-ene-DON) and/or DON derivative/s (preferably, said modifying comprising changing a hydroxyl moiety (e.g., —OH or C—OH) to carbonyl moiety (e.g., can be referred to as ═O or C═O) at position 7 of the carbon chain; further preferably said DON derivative is not 3-Ac-DON), said method comprising: (i) providing one or more of the following polypeptides: (a) a polypeptide comprising the amino acid sequence set forth in SEQ ID NO: 1; (b) a polypeptide comprising an amino acid sequence having at least 70% (e.g., at least 80%, at least 85%, at least 90%, at least 91%, at least 92%, at least 93%, at least 94%, at least 95%, at least 96%, at least 97%, at least 98%, at least 99%) identity to the amino acid sequence set forth in SEQ ID NO: 1; preferably said polypeptide is not having 100% identity to SEQ ID NO: 2-3; (c) a variant of the polypeptide set forth in SEQ ID NO: 1, wherein said variant comprising: a substitution, deletion, and/or insertion at one or more positions; preferably said variant is not having 100% identity to SEQ ID NO: 2-3 and/or (d) a fragment of the polypeptide of (a), (b) or (c), wherein said fragment is capable of degrading DON (e.g., by producing 7-one-8-hydroxy-8-ene-DON) and/or DON derivative/s and/or altering toxicity of DON (e.g., by producing 7-one-8-hydroxy-8-ene-DON) and/or DON derivatives, preferably said altering toxicity comprising one or more of the following: detoxifying and/or changing intermolecular rearrangement of DON (e.g., by producing 7-one-8-hydroxy-8-ene-DON) and/or DON derivatives (preferably said DON derivative is not 3-Ac-DON); (ii) applying one or more of (a)-(d) to DON and/or DON derivative/s; preferably said one or more polypeptides are one or more recombinant and/or isolated polypeptides;

In some embodiments, the method of the present invention is an in vitro, ex vivo or in vivo method.

In some embodiments, the present invention relates to the use of one or more of the following: (a) a polypeptide comprising the amino acid sequence set forth in SEQ ID NO: 1; (b) a polypeptide comprising an amino acid sequence having at least 70% (e.g., at least 80%, at least 85%, at least 90%, at least 91%, at least 92%, at least 93%, at least 94%, at least 95%, at least 96%, at least 97%, at least 98%, at least 99%) identity to the amino acid sequence set forth in SEQ ID NO: 1; preferably said polypeptide is not having 100% identity to SEQ ID NO: 2-3; (c) a variant of the polypeptide set forth in SEQ ID NO: 1, wherein said variant comprising: a substitution, deletion, and/or insertion at one or more positions; and/or (d) a fragment of the polypeptide of (a), (b) or (c); (e) composition, kit, foodstuff, intermediate foodstuff; fodder, intermediate fodder; feed, intermediate feed; additive (e.g., foodstuff-, fodder- or feed additive, processing aid, reactant or catalyst, e.g., in food or feed industry, citric acid, bioethanol, starch production etc.), intermediate additive (e.g., foodstuff-, fodder- or feed intermediate additive, intermediate processing aid, reactant or catalyst, e.g., in food or feed industry, citric acid, bioethanol, starch production etc.); detoxifying agent, intermediate detoxifying agent; nutritional supplement, intermediate nutritional supplement; prebiotic, intermediate prebiotic or mixture/s thereof of the present invention for/in one or more of the following: (i) modifying the C7-atom of DON (e.g., by producing 7-one-8-hydroxy-8-ene-DON) and/or DON derivative/s (preferably, said modifying comprising changing a hydroxyl moiety (—OH) to carbonyl moiety (═O) at C7; further preferably said DON derivative is not 3-Ac-DON); (ii) producing a foodstuff, intermediate foodstuff; fodder, intermediate fodder; feed, intermediate feed; additive (e.g., foodstuff-, fodder- or feed additive processing aid, reactant or catalyst, e.g., in food or feed industry, citric acid, bioethanol, starch production etc.), intermediate additive (e.g., foodstuff-, fodder- or feed intermediate additive, intermediate processing aid, reactant or catalyst, e.g., in food or feed industry, citric acid, bioethanol, starch production etc.); detoxifying agent, intermediate detoxifying agent; nutritional supplement, intermediate nutritional supplement; prebiotic, intermediate prebiotic and/or mixture/s thereof; (iii) degrading DON and/or DON derivative/s and/or altering toxicity of DON and/or DON derivative/s, preferably said one or more polypeptides are recombinant and/or isolated polypeptides; further preferably said altering toxicity comprising one or more of the following: detoxifying and/or intramolecularly rearranging DON (e.g., by producing 7-one-8-hydroxy-8-ene-DON) and/or DON derivative/s (preferably, said modifying comprising changing a hydroxyl moiety (—OH) to carbonyl moiety (═O) at C7; further preferably said DON derivative is not 3-Ac-DON); most preferably said one or more polypeptides are capable of modifying the C7-atom of DON and/or DON derivatives (preferably, said modifying comprising changing a hydroxyl moiety (—OH) to carbonyl moiety (═O) at C7; further preferably said DON derivative is not 3-Ac-DON); (iv) any combination of (i)-(iii); (v) use according to any one (i)-(iv), wherein said use is an in vitro, ex vivo or in vivo use.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The invention is also characterized by the following items:
1. A method for producing a foodstuff, intermediate foodstuff; fodder, intermediate fodder; feed, intermediate feed; additive (e.g., foodstuff-, fodder- or feed additive, processing aid, reactant or catalyst, e.g., in food or feed industry, citric acid, bioethanol, starch production etc.), intermediate additive (e.g., foodstuff-, fodder- or feed intermediate additive, intermediate processing aid, reactant or catalyst, e.g., in food or feed industry, citric acid, bioethanol, starch production etc.); detoxifying agent, intermediate detoxifying agent; nutritional supplement, intermediate nutritional supplement; prebiotic, intermediate prebiotic and/or mixture/s thereof, said method comprising:
   i) providing one or more of the following polypeptides:
      (a) a polypeptide comprising the amino acid sequence set forth in SEQ ID NO: 1
      (b) a polypeptide comprising an amino acid sequence having at least 70% (e.g., at least 80%, at least 85%, at least 90%, at least 91%, at least 92%, at least 93%, at least 94%, at least 95%, at least 96%, at least 97%, at least 98%, at least 99%) identity to the amino acid sequence set forth in SEQ ID NO: 1; preferably said polypeptide does not have a 100% identity amino acid sequence set forth in SEQ ID NOs: 2-3;
      (c) a variant of the polypeptide set forth in SEQ ID NO: 1, wherein said variant comprising: a substitution, deletion, and/or insertion at one or more positions; preferably said variant does not have a 100% identity to amino acid sequence set forth in SEQ ID NOs: 2-3; and/or
      (d) a fragment of the polypeptide of (a), (b) or (c), wherein said fragment is capable of degrading DON (e.g., to 7-one-8-hydroxy-8-ene-DON) and/or DON derivatives (e.g., DON derivative as used herein may comprise a hydroxyl moiety (—OH) at the C7-atom, a carbonyl moiety (═O) at C8; C10 not substituted) and/or altering toxicity of DON (e.g., by producing 7-one-8-hydroxy-8-ene-DON) and/or DON derivative/s (e.g., DON derivative may comprise a hydroxyl moiety (—OH) at the C7-atom, a carbonyl moiety (e.g., ═O) at C8; C10 not substituted), preferably said altering toxicity comprising one or more of the following: detoxifying and/or changing intermolecular rearrangement of DON (e.g., by producing 7-one-8-hydroxy-8-ene-DON) and/or DON derivatives;
   ii) applying one or more of (a)-(d) to a nutritive source (e.g., comprising a carbohydrate and/or protein source) or material (e.g., raw material, crop, grain, citric acid, bioethanol, etc.) suitable for production of foodstuff, intermediate foodstuff; fodder, intermediate fodder; feed, intermediate feed; additive (e.g., foodstuff-, fodder- or feed additive, processing aid, reactant or catalyst, e.g., in food or feed industry, citric acid, bioethanol, starch production etc.), intermediate additive (e.g., foodstuff-, fodder- or feed intermediate additive, intermediate processing aid, reactant or catalyst, e.g., in food or feed industry, citric acid, bioethanol, starch production etc.); detoxifying agent, intermediate detoxifying agent; nutritional supplement, intermediate nutritional supplement; prebiotic, intermediate prebiotic and/or mixture/s thereof;
   preferably said one or more polypeptides are one or more recombinant and/or isolated polypeptides; further preferably said one or more polypeptides are capable of modifying the C7-atom of DON (e.g., by producing 7-one-8-hydroxy-8-ene-DON) and/or DON derivative/s; most preferably said modifying comprising changing a hydroxyl-moiety (e.g., C—OH) to carbonyl moiety (e.g., C═O) at C7-atom; further most preferably said DON derivative is not 3-Ac-DON, i.e., 3-Acetyl oxy-3α,7α-dihydroxy-12,13-epoxytrichothec-9-en-8-one).
2. The method according any one of the preceding items, said method further comprising: incubating said nutritive source or material with one or more of (a)-(d) under conditions suitable for degrading DON and/or DON derivative/s and/or altering toxicity of DON and/or DON derivative/s; preferably said method further comprising heat-treating and/or fractionating and/or drying the product of said incubation; further preferably said altering toxicity comprising one or more of the following: detoxifying (e.g., by producing 7-one-8-hydroxy-8-ene-DON) and/or intramolecularly rearranging DON (e.g., by producing 7-one-8-hydroxy-8-ene-DON) and/or DON derivative/s (preferably said DON derivative is not 3-Ac-DON, i.e., 3-Acetyl oxy-3α,7α-dihydroxy-12,13-epoxytrichothec-9-en-8-one).
3. A foodstuff, intermediate foodstuff; fodder, intermediate fodder; feed, intermediate feed; additive (e.g., foodstuff-, fodder- or feed additive, processing aid, reactant or catalyst, e.g., in food or feed industry, citric acid, bioethanol, starch production etc.), intermediate additive (e.g., foodstuff-, fodder- or feed intermediate additive, intermediate processing aid, reactant or catalyst, e.g., in food or feed industry, citric acid, bioethanol, starch production etc.); detoxifying agent, intermediate detoxifying agent; nutritional supplement, intermediate nutritional supplement; prebiotic, intermediate prebiotic and/or mixture/s thereof produced by the method according to any one of preceding items.
4. A method for degrading DON (e.g., by producing 7-one-8-hydroxy-8-ene-DON) and/or DON derivative/s and/or altering toxicity of DON (e.g., by producing 7-one-8-hydroxy-8-ene-DON) and/or DON derivative/s (e.g., DON derivative may comprise a hydroxyl moiety (—OH) at the C7-atom, a carbonyl moiety (═O) at C8; C10 not substituted; said degrading and/or altering toxicity comprising: modifying the C7-atom of DON and/or DON derivative/s; preferably said DON derivative is not 3-Ac-DON, i.e., 3-Acetyloxy-3α,7α-dihydroxy-12,13-epoxytrichothec-9-en-8-one), said method comprising:
   i) providing one or more of the following polypeptides:
      (a) a polypeptide comprising the amino acid sequence set forth in SEQ ID NO: 1;
      (b) a polypeptide comprising an amino acid sequence having at least 70% (e.g., at least 80%, at least 85%, at least 90%, at least 91%, at least 92%, at least 93%, at least 94%, at least 95%, at least 96%, at least 97%, at least 98%, at least 99%) identity to the amino acid sequence set forth in SEQ ID NO: 1, preferably said polypeptide is not having 100% identity to SEQ ID NO: 2-3;
      (c) a variant of the polypeptide set forth in SEQ ID NO: 1, wherein said variant comprising: a substitution, deletion, and/or insertion at one or more positions; preferably said variant is not having 100% identity to SEQ ID NO: 2-3; and/or
      (d) a fragment of the polypeptide of (a), (b) or (c), wherein said fragment is capable of degrading DON (e.g., by producing 7-one-8-hydroxy-8-ene-DON) and/or DON derivative/s and/or altering toxicity of DON (e.g., by producing 7-one-8-hydroxy-8-ene-DON) and/or DON derivative/s, preferably said altering toxicity comprising one or more of the following: detoxifying and/or changing intermolecular rearrangement of DON (e.g., by producing 7-one-8-hydroxy-8-ene-DON) and/or DON derivative/s;
   ii) applying one or more of (a)-(d) to DON and/or DON derivative/s;
   preferably said one or more polypeptides are one or more recombinant and/or isolated polypeptides; further preferably said altering toxicity comprising one or more of the following: detoxifying, and/or intramolecularly rearranging DON (e.g., by producing 7-one-8-hydroxy-8-ene-DON) and/or DON derivative/s (preferably said DON derivative is not 3-Ac-DON, i.e., 3-Acetyloxy-3α,7α-dihydroxy-12,13-epoxytrichothec-9-en-8-one)); most preferably said one or more polypeptides are capable of modifying bonds at the C7-atom of DON and/or DON derivatives (e.g., said modifying comprising changing C—OH to C═O moiety, e.g., at position 7; preferably said DON derivative is not 3-Ac-DON, i.e., 3-Acetyloxy-3α,7α-dihydroxy-12,13-epoxytrichothec-9-en-8-one).
5. A composition or kit, comprising:
   (i) a polypeptide capable of degrading DON (e.g., by producing 7-one-8-hydroxy-8-ene-DON) and/or DON derivative/s (e.g., DON derivative may comprise a hydroxyl moiety (—OH) at the C7-atom, a carbonyl moiety (═O) at C8; C10 not substituted; preferably said DON derivative is not 3-Ac-DON, i.e., 3-Acetyloxy-3α,7α-dihydroxy-12,13-epoxytrichothec-9-en-8-one) and/or altering toxicity of DON (e.g., by producing 7-one-8-hydroxy-8-ene-DON) and/or DON derivative/s, wherein said polypeptide is one or more of the following:
      (a) a polypeptide comprising the amino acid sequence set forth in SEQ ID NO: 1;
      (b) a polypeptide comprising an amino acid sequence having at least 70% (e.g., at least 80%, at least 85%, at least 90%, at least 91%, at least 92%, at least 93%, at least 94%, at least 95%, at least 96%, at least 97%, at least 98%, at least 99%) identity to the amino acid sequence set forth in SEQ ID NO: 1; preferably said polypeptide is not having 100% identity to SEQ ID NO: 2-3;
      (c) a variant of the polypeptide set forth in SEQ ID NO: 1, wherein said variant comprising: a substitution, deletion, and/or insertion at one or more positions; preferably said variant is not having 100% identity to SEQ ID NO: 2-3; and/or
      (d) a fragment of the polypeptide of (a), (b) or (c), wherein said fragment is capable of degrading DON (e.g., by producing 7-one-8-hydroxy-8-ene-DON) and/or DON derivatives and/or altering toxicity of DON (e.g., by producing 7-one-8-hydroxy-8-ene-DON) and/or DON derivatives, preferably said altering toxicity comprising one or more of the following: detoxifying and/or changing intermolecular rearrangement of DON (e.g., by producing 7-one-8-hydroxy-8-ene-DON) and/or DON derivative/s (preferably said DON derivative is not 3-Ac-DON, i.e., 3-Acetyloxy-3α,7α-dihydroxy-12,13-epoxytrichothec-9-en-8-one);
   (ii) a nutritive source (e.g., comprising one or more of the following: carbohydrate, protein, citric acid, ethanol, e.g., bioethanol; preferably said nutritive source is not 3-Ac-DON) and/or a substrate for at least one of the polypeptides (a)-(d), preferably said substrate comprising: DON and/or DON derivative/s (preferably, said substrate is not 3-Ac-DON; further preferably said composition further comprising the product of the reaction (e.g., enzymatic reaction) of said one or more polypeptides with DON (e.g., 7-one-8-hydroxy-8-ene-DON) and/or DON derivative/s, i.e., a reaction product comprising a modification at the C7-atom of DON and/or DON derivative/s);
   preferably said polypeptide is a recombinant and/or isolated polypeptide; further preferably said altering toxicity comprising one or more of the following: detoxifying, and/or intramolecularly rearranging DON and/or DON derivative/s (preferably, DON derivative is not 3-Ac-DON); most preferably said polypeptide is capable of modifying the C7-atom of DON and/or DON derivatives (preferably, said modifying comprising changing C—OH to C═O moiety; further preferably said DON derivative is not 3-Ac-DON).
6. The composition or kit according to any one of the preceding items, wherein said one or more polypeptides are encoded by one or more nucleotide sequences.
7. The composition or kit according to any one of the preceding items, wherein said one or more nucleotide sequences are comprised by one or more nucleic acids comprised by a host cell.
8. The composition or kit according to any one of the preceding items, wherein said composition or kit is one or more of the following: cell-free and/or non-naturally occurring and/or fractionated composition or kit.
9. The composition or kit according to any one of the preceding items, wherein said composition or kit is a pharmaceutical or veterinary composition or kit.
10. The composition, kit, foodstuff, intermediate foodstuff; fodder, intermediate fodder; feed, intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement; prebiotic, intermediate prebiotic or mixture/s thereof according to any one of the preceding items, for use as a medicament (e.g., for veterinary use) and/or in therapy.
11. The composition, kit, foodstuff, intermediate foodstuff; fodder, intermediate fodder; feed, intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement; prebiotic, intermediate prebiotic or mixture/s thereof according to any one of the preceding items, for use in treatment, amelioration, prophylaxis and/or diagnostics of DON mycotoxicosis.
12. A method for modifying the C7-atom of DON (e.g., by producing 7-one-8-hydroxy-8-ene-DON) and/or DON derivative/s (preferably, said modifying comprising changing a hydroxyl moiety (—OH) to carbonyl moiety (═O) at C7; further preferably said DON derivative is not 3-Ac-DON), said method comprising:
   i) providing one or more of the following polypeptides:
      (a) a polypeptide comprising the amino acid sequence set forth in SEQ ID NO: 1;
      (b) a polypeptide comprising an amino acid sequence having at least 70% (e.g., at least 80%, at least 85%, at least 90%, at least 91%, at least 92%, at least 93%, at least 94%, at least 95%, at least 96%, at least 97%, at least 98%, at least 99%) identity to the amino acid sequence set forth in SEQ ID NO: 1; preferably said polypeptide is not having 100% identity to SEQ ID NO: 2-3
      (c) a variant of the polypeptide set forth in SEQ ID NO: 1, wherein said variant comprising: a substitution, deletion, and/or insertion at one or more positions; preferably said variant is not having 100% identity to SEQ ID NO: 2-3 and/or
      (d) a fragment of the polypeptide of (a), (b) or (c), wherein said fragment is capable of degrading DON (e.g., by producing 7-one-8-hydroxy-8-ene-DON) and/or DON derivative/s and/or altering toxicity of DON (e.g., by producing 7-one-8-hydroxy-8-ene-DON) and/or DON derivatives, preferably said altering toxicity comprising one or more of the following: detoxifying and/or changing intermolecular rearrangement of DON (e.g., by producing 7-one-8-hydroxy-8-ene-DON) and/or DON derivatives (preferably said DON derivative is not 3-Ac-DON);
   ii) applying one or more of (a)-(d) to DON and/or DON derivative/s; preferably said one or more polypeptides are one or more recombinant and/or isolated polypeptides;
13. The method according to any one of the preceding items, wherein said method is an in vitro, ex vivo or in vivo method.
14. Use of one or more of the following:
   (a) a polypeptide comprising the amino acid sequence set forth in SEQ ID NO: 1;
   (b) a polypeptide comprising an amino acid sequence having at least 70% (e.g., at least 80%, at least 85%, at least 90%, at least 91%, at least 92%, at least 93%, at least 94%, at least 95%, at least 96%, at least 97%, at least 98%, at least 99%) identity to the amino acid sequence set forth in SEQ ID NO: 1; preferably said polypeptide is not having 100% identity to SEQ ID NO: 2-3;
   (c) a variant of the polypeptide set forth in SEQ ID NO: 1, wherein said variant comprising: a substitution, deletion, and/or insertion at one or more positions; and/or
   (d) a fragment of the polypeptide of (a), (b) or (c);
   (e) composition, kit, foodstuff, intermediate foodstuff; fodder, intermediate fodder; feed, intermediate feed; additive (e.g., foodstuff-, fodder- or feed additive, processing aid, reactant or catalyst, e.g., in food or feed industry, citric acid, bioethanol, starch production etc.), intermediate additive (e.g., foodstuff-, fodder- or feed intermediate additive, intermediate processing aid, reactant or catalyst, e.g., in food or feed industry, citric acid, bioethanol, starch production etc.); detoxifying agent, intermediate detoxifying agent; nutritional supplement, intermediate nutritional supplement; prebiotic, intermediate prebiotic or mixture/s thereof according to any one of the preceding items;
   for/in one or more of the following:
   i) modifying the C7-atom of DON (e.g., by producing 7-one-8-hydroxy-8-ene-DON) and/or DON derivative/s (preferably, said modifying comprising changing a hydroxyl moiety (—OH) to carbonyl moiety (═O) at C7; further preferably said DON derivative is not 3-Ac-DON);
   ii) producing a foodstuff, intermediate foodstuff; fodder, intermediate fodder; feed, intermediate feed; additive (e.g., foodstuff-, fodder- or feed additive), intermediate additive (e.g., foodstuff-, fodder- or feed intermediate additive); detoxifying agent, intermediate detoxifying agent; nutritional supplement, intermediate nutritional supplement; prebiotic, intermediate prebiotic and/or mixture/s thereof;
   iii) degrading DON and/or DON derivative/s and/or altering toxicity of DON and/or DON derivative/s, preferably said one or more polypeptides are recombinant and/or isolated polypeptides; further preferably said altering toxicity comprising one or more of the following: detoxifying and/or intramolecularly rearranging DON (e.g., by producing 7-one-8-hydroxy-8-ene-DON) and/or DON derivative/s (preferably, said modifying comprising changing a hydroxyl moiety (—OH) to carbonyl moiety (═O) at C7; further preferably said DON derivative is not 3-Ac-DON); most preferably said one or more polypeptides are capable of modifying the C7-atom of DON and/or DON derivatives (preferably, said modifying comprising changing a hydroxyl moiety (—OH) to carbonyl moiety (═O) at C7; further preferably said DON derivative is not 3-Ac-DON);
   iv) any combination of (i)-(iii);
   v) use according to any one (i)-(iv), wherein said use is an in vitro, ex vivo or in vivo use.

The invention is further illustrated by the following examples, however, without being limited to the example or by any specific embodiment of the examples.

### EXAMPLES OF THE INVENTION

### Example 1: Recombinant Production of Polypeptides

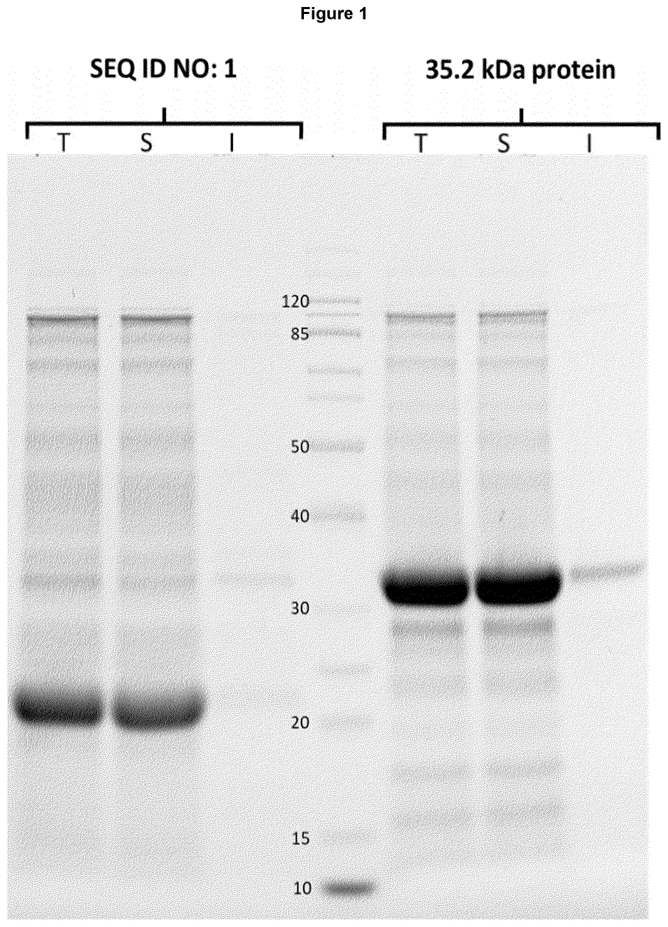

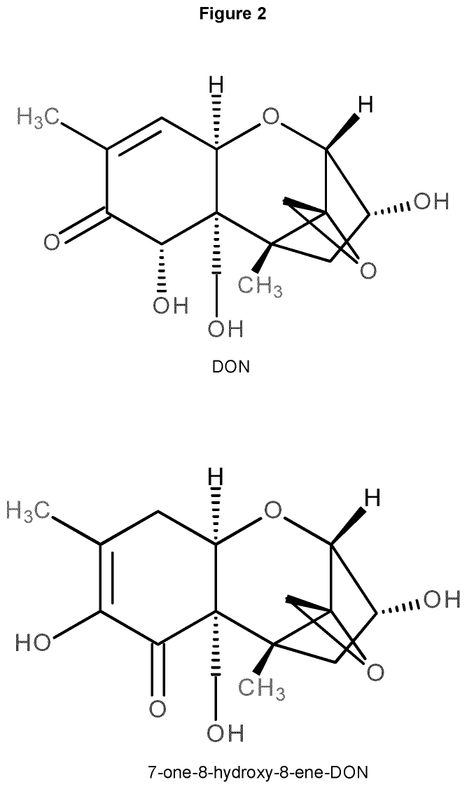

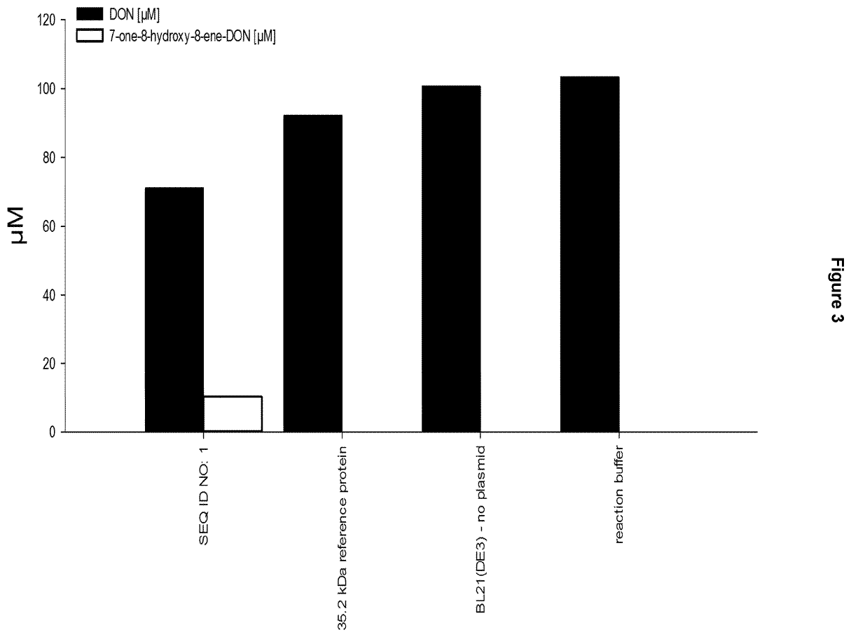

Genes for the polypeptide sequences (SEQ ID NO: 1, SEQ ID NO: 2 and SEQ ID NO: 3) were synthesized by the commercial provider TWIST Bioscience (https://www.twistbioscience.com/) with codons optimized for expression in E. coli. The genes were inserted into the plasmid vector pE2 for expression under control of the T7lac promoter (Dubendorf and Studier (1991), J. Mol. Biol. 219, 45-59). The generated plasmids were transformed into E. coli BL21(DE3) by using a modification of a heat-shock procedure (Cohen et al. (1972), Proc. Natl. Acad. Sci. 69, 2110-2114) and by selection for plasmid-mediated kanamycin resistance. Plasmid-harboring E. coli BL21(DE3) clones were cultivated in Overnight Express Instant TB Medium (Merck) with shaking at 30° C. over night for recombinant gene expression by autoinduction (Studier (2005), Protein Expr. Purif. 41, 207-234). Biomass was harvested by centrifugation, resuspended in 1.1×reaction buffer (reaction buffer: 25 mM Hepes pH 7.5; 10 mM ZnCl₂), and lysed with an ultrasonication system (QSonica) on ice. Crude lysate was cleared by centrifugation (18 min 21 130 rcf 4° C.), and presence of polypeptide (enzyme) in the soluble fraction of lysate was verified by SDS-PAGE (Laemmli (1970), Nature 227, 680-685) with Bio-Rad 12% mini-PROTEAN TGX stain-free precast gels (FIG. 1).

### Example 2: Analytical Quantification of DON and 7-one-8-hydroxy-8-ene-DON

DON and the reaction product of enzyme activity of SEQ ID NO: 1 were separated by HPLC on a Phenomenex Kinetex C18 column (150×2.1 mm, 2.6 μm particle size) at 30° C. in a gradient from 5.9% to 95% acetonitrile with 0.1% acetic acid. The reaction product was identified by comparison with a purified reference substance, and named 7-one-8-hydroxy-8-ene-DON, following the atom numbering proposed by Yoshizawa and Morooka in the first description of DON (Yoshizama and Morooka (1973), Agric. Biol. Chem. 37, 2933-2934). Quantification was based on MS/MS with a Sciex QTRAP 5500 System in negative multiple reaction monitoring (MRM) mode and comparison with pure reference preparations of DON and 7-one-8-hydroxy-8-ene-DON. Mass transition from precursor ion [M+Ac]⁻ with m/z 355.1 to product ion 59.1 was used as quantifier, and transition from m/z 355.1 to 265.1 was used as qualifier.

### Example 3: DON Modification/Detoxification Activity of Polypeptides

1 mM DON in water was added to crude E. coli cell lysate, prepared as described in Example 1, to start reactions in buffer with 25 mM Hepes pH 7.5, 10 mM ZnCl₂, and 100 μM DON. After 3 h incubation at 30° C., reactions were stopped by freezing at −20° C. To process reaction end-point samples for HPLC analysis, reactions were thawed, vortexed, and 10 μl per reaction was added to 40 μl water and 200 μl acetonitrile. These samples with 80% acetonitrile and nominally 4 μM DON were incubated at room temperature for 10 min, centrifuged at 21 130 rcf for 10 min, and 250 μl supernatant was added to 750 μl water. Such diluted samples with 20% acetonitrile and nominally 1 μM DON were centrifuged again for 10 min at 21 130 rcf, and 500 μl was transferred to HPLC vials for analysis. The polypeptide SEQ ID NO: 1 was able to convert DON to 7-one-8-hydroxy-8-ene-DON (quantification is shown in FIG. 3), whereas SEQ ID NO: 2 and SEQ ID NO: 3 did not show any conversion of DON or any reduction of DON concentration. It is well known in the art, that 7-one-8-hydroxy-8-ene-DON is less toxic than DON (Stadler et al. (2019), Food Chem. 279, 303-311; Stadler et al. (2019), Toxins (Basel) 11, 317). Thus, it was surprisingly found that SEQ ID NO: 1 is able to detoxify DON.

### Example 4: Enzymatic Kinetics of DON-Conversion

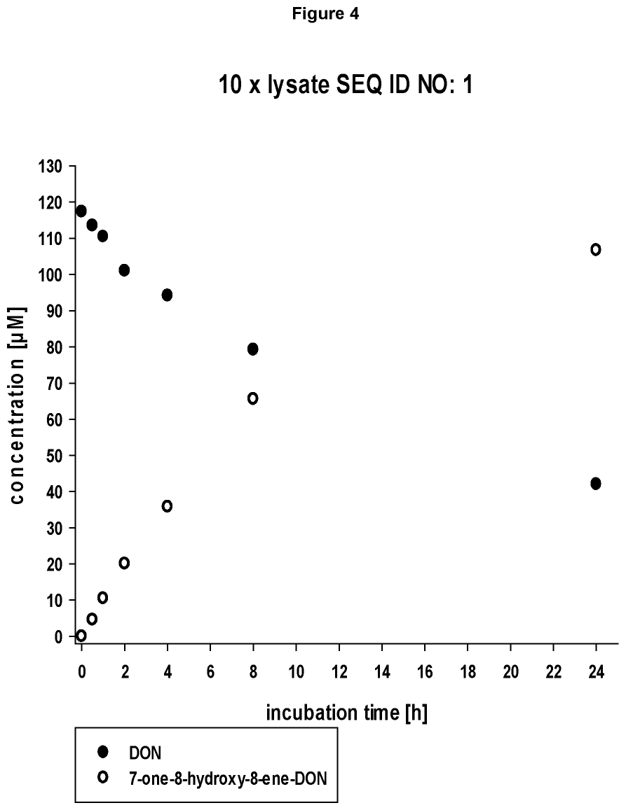

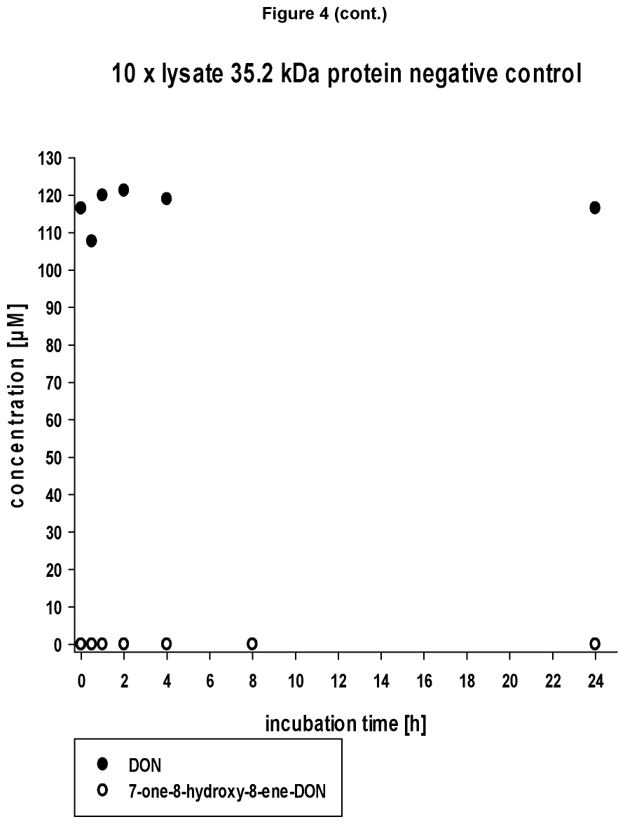

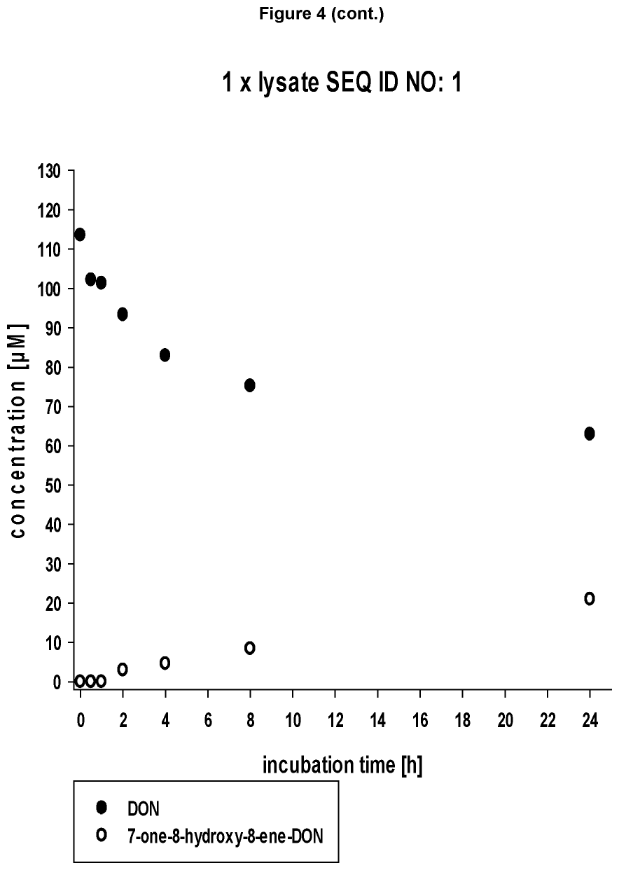

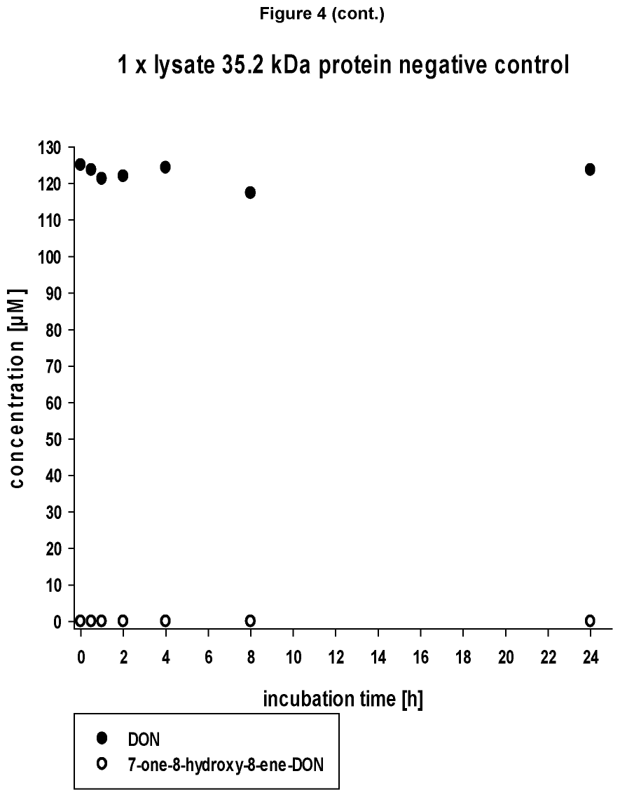

The polypeptide SEQ ID NO: 1 and a 35.2 kDa reference protein (negative control) were produced by gene expression in E. coli BL21(DE3) in Overnight Express Instant TB Medium (Merck) as described in Example 1. Biomass from the same culture volume or the 10-fold culture volume was resuspended in 1.1×reaction buffer (reaction buffer: 25 mM Hepes pH 7.5; 10 mM ZnCl₂), and lysed on ice with an ultrasonication system (QSonica). Lysates were cleared by centrifugation (20 min 30 272 rcf 4° C.), and presence of recombinant protein in clear lysate was verified by SDS-PAGE. Reactions were started by addition of 1 mM DON to a final concentration of 100 μM DON in 25 mM Hepes pH 7.5 with 10 mM ZnCl₂, and incubated at 30° C. Time point samples were taken and inactivated by addition of 10 μl sample to 40 μl water and 200 μl acetonitrile. Such stopped samples were centrifuged (20 min 30 272 rcf 4° C.), and 250 μl supernatant was transferred to 750 μl water to reach a final nominal DON concentration of 1 μM in 20% acetonitrile. After centrifugation (1 min 21 130 rcf 4° C.), 500 μl was transferred to HPLC vials for analysis. Time courses of concentrations of DON and 7-one-8-hydroxy-8-ene-DON are shown in FIG. 4.

1. A method for producing a foodstuff, intermediate foodstuff; fodder, intermediate fodder, feed, intermediate feed, additive, intermediate additive, detoxifying agent, intermediate detoxifying agent, nutritional supplement, intermediate nutritional supplement, prebiotic, intermediate prebiotic and/or mixture/s thereof, said method comprising:
i) providing one or more of the following polypeptides, wherein said one or more polypeptides are capable of modifying the C7-atom of deoxynivalenol (DON) and/or DON derivative selected from the group consisting of: 3-keto Deoxynivalenol, 3-epi Deoxynivalenol, 15-Acetyldemrynivalenol, Nivalenol, Fusarenon-X (4-acetylnivalenol), 3-amino Deoxynivalenol:
(a) a polypeptide comprising the amino acid sequence set forth in SEQ ID NO: 1;
(b) a polypeptide comprising an amino acid sequence having at least 70% (e.g., at least 95%, at least 96%, at least 97%, at least 98% or at least 99%) identity to the amino acid sequence set forth in SEQ ID NO: 1, but less than 100% sequence identity with the amino acid sequence set forth in SEQ ID NOs: 2 or 3, wherein said polypeptide is capable of modifying the C7-atom of deoxynivalenol (DON) and/or DON derivative selected from the group consisting of: 3-keto Deoxynivalenol, 3-epi Deoxynivalenol, 15-Acetyldeoxynivalenol, Nivalenol, Fusarenon-X (4-acetylnivalenol), 3-amino Deoxynivalenol;
ii) applying one or more of (a)-(b) to a nutritive source or material suitable for production of foodstuff, intermediate foodstuff, fodder, intermediate fodder, feed, intermediate feed, additive, intermediate additive, detoxifying agent, intermediate detoxifying agent, nutritional supplement, intermediate nutritional supplement, prebiotic, intermediate prebiotic and/or mixture/s thereof;
preferably said one or more polypeptides are one or more recombinant and/or isolated polypeptides; further preferably said one or more polypeptides are capable of modifying the C7-atom of DON and/or DON derivative/s.

2. The method according to any one of the preceding claims, said method further comprising: incubating said nutritive source or material with one or more of (a)-(b) under conditions suitable for degrading DON and/or said DON derivative/s and/or altering toxicity of DON and/or said DON derivative/s; preferably said method further comprising heat-treating and/or fractionating and/or drying the product of said incubation; further preferably said altering toxicity comprising one or more of the following: detoxifying, and/or intramolecularly rearranging DON and/or said DON derivative/s.

3. A foodstuff, intermediate foodstuff; fodder, intermediate fodder; feed, intermediate feed, additive, intermediate additive, detoxifying agent, intermediate detoxifying agent, nutritional supplement, intermediate nutritional supplement, prebiotic, intermediate prebiotic and/or mixture/s thereof produced by the method according to any one of the preceding claims, wherein said foodstuff, intermediate foodstuff; fodder, intermediate fodder; feed, intermediate feed, additive, intermediate additive, detoxifying agent, intermediate detoxifying agent, nutritional supplement, intermediate nutritional supplement, prebiotic, intermediate prebiotic and/or mixture/s thereof comprising said one or more polypeptides according to claim 1.

4. A method for degrading deoxynivalenol (DON) and/or DON derivative selected from the group consisting of: 3-keto Deoxynivalenol, 3-epi Deoxynivalenol, 15-Acetyldeoxynivalenol, Nivalenol, Fusarenon-X (4-acetylnivalenol), 3-amino Deoxynivalenol and/or altering toxicity of DON and/or said DON derivative/s, said method comprising:
i) providing one or more of the following polypeptides, wherein said one or more polypeptides are capable of modifying the C7-atom of deoxynivalenol (DON) and/or DON derivative selected from the group consisting of: 3-keto Deoxynivalenol, 3-epi Deoxynivalenol, 15-Acetyldemrynivalenol, Nivalenol, Fusarenon-X (4-acetylnivalenol), 3-amino Deoxynivalenol:
(a) a polypeptide comprising the amino acid sequence set forth in SEQ ID NO: 1;
(b) a polypeptide comprising an amino acid sequence having at least 70% (e.g., at least 95%, at least 96%, at least 97%, at least 98% or at least 99%) identity to the amino acid sequence set forth in SEQ ID NO: 1, but less than 100% sequence identity with the amino acid sequence set forth in SEQ ID NOs: 2 or 3, wherein said polypeptide is capable of modifying the C7-atom of deoxynivalenol (DON) and/or DON derivative selected from the group consisting of: 3-keto Deoxynivalenol, 3-epi Deoxynivalenol, 15-Acetyldeoxynivalenol, Nivalenol, Fusarenon-X (4-acetylnivalenol), 3-amino Deoxynivalenol;
ii) applying one or more of (a)-(b) to DON and/or said DON derivative/s;
preferably said one or more polypeptides are one or more recombinant and/or isolated polypeptides; further preferably said altering toxicity comprising one or more of the following: detoxifying and/or intramolecularly rearranging DON and/or DON derivative/s; most preferably said one or more polypeptides are capable of modifying the C7-atom of DON and/or DON derivative/s.

5. A composition or kit, comprising:
(i) a polypeptide capable of degrading deoxynivalenol (DON) and/or DON derivative selected from the group consisting of: 3-keto Deoxynivalenol, 3-epi Deoxynivalenol, 15-Acetyldeoxynivalenol, Nivalenol, Fusarenon-X (4-acetylnivalenol), 3-amino Deoxynivalenol and/or altering toxicity of DON and/or said DON derivative/s, wherein said polypeptide is one or more of the following:
(a) a polypeptide comprising the amino acid sequence set forth in SEQ ID NO: 1;
(b) a polypeptide comprising an amino acid sequence having at least 70% (e.g., at least 95%, at least 96%, at least 97%, at least 98% or at least 99%) identity to the amino acid sequence set forth in SEQ ID NO: 1, but less than 100% sequence identity with the amino acid sequence set forth in SEQ ID NOs: 2 or 3, wherein said polypeptide is capable of modifying the C7-atom of deoxynivalenol (DON) and/or DON derivative selected from the group consisting of: 3-keto Deoxynivalenol, 3-epi Deoxynivalenol, 15-Acetyldeoxynivalenol, Nivalenol, Fusarenon-X (4-acetylnivalenol), 3-amino Deoxynivalenol;
(ii) a nutritive source and/or a substrate for at least one of the polypeptides (a)-(b), preferably said nutritive source and/or substrate comprising DON and/or said DON derivative/s;
preferably said polypeptide is a recombinant and/or isolated polypeptide; further preferably said altering toxicity comprising one or more of the following: detoxifying and/or intramolecularly rearranging DON and/or DON derivative/s; most preferably said polypeptide is capable of modifying the C7-atom of DON and/or DON derivative/s.

6. The composition or kit according to any one of the preceding claims, wherein said one or more polypeptides are encoded by one or more nucleotide sequence/s.

7. The composition or kit according to any one of the preceding claims, wherein said one or more nucleotide sequences are comprised by one or more nucleic acids comprised by a host cell.

8. The composition or kit according to any one of the preceding claims, wherein said composition or kit is one or more of the following: cell-free and/or non-naturally occurring and/or fractionated composition or kit.

9. The composition or kit according to any one of the preceding claims, wherein said composition or kit is a pharmaceutical and/or veterinary composition or kit.

10. The composition, kit, foodstuff, intermediate foodstuff; fodder, intermediate fodder; feed, intermediate feed, additive, intermediate additive, detoxifying agent, intermediate detoxifying agent, nutritional supplement, intermediate nutritional supplement, prebiotic, intermediate prebiotic or mixture/s thereof according to any one of the preceding claims, for use as a medicament (e.g., for veterinary use) and/or in therapy.

11. The composition, kit, foodstuff, intermediate foodstuff; fodder, intermediate fodder; feed, intermediate feed, additive, intermediate additive, detoxifying agent, intermediate detoxifying agent, nutritional supplement, intermediate nutritional supplement, prebiotic, intermediate prebiotic or mixture/s thereof according to any one of the preceding claims, for use in treatment, amelioration, prophylaxis and/or diagnostics of DON mycotoxicosis.

12. A method for modifying the C7-atom of deoxynivalenol (DON) and/or DON derivative selected from the group consisting of: 3-keto Deoxynivalenol, 3-epi Deoxynivalenol, 15-Acetyldeoxynivalenol, Nivalenol, Fusarenon-X (4-acetylnivalenol), 3-amino Deoxynivalenol, said method comprising:
i) providing one or more of the following polypeptides:
(a) a polypeptide comprising the amino acid sequence set forth in SEQ ID NO: 1;
(b) a polypeptide comprising an amino acid sequence having at least 70% (e.g., at least 95%, at least 96%, at least 97%, at least 98% or at least 99%) identity to the amino acid sequence set forth in SEQ ID NO: 1, but less than 100% sequence identity with the amino acid sequence set forth in SEQ ID NOs: 2 or 3, wherein said polypeptide is capable of modifying the C7-atom of deoxynivalenol (DON) and/or DON derivative selected from the group consisting of: 3-keto Deoxynivalenol, 3-epi Deoxynivalenol, 15-Acetyldeoxynivalenol, Nivalenol, Fusarenon-X (4-acetylnivalenol), 3-amino Deoxynivalenol;
ii) applying one or more of (a)-(b) to DON and/or said DON derivative/s;
preferably said one or more polypeptides are one or more recombinant and/or isolated polypeptides.

13. The method according to any one of the preceding claims, wherein said method is an in vitro, ex vivo or in vivo method.

14. Use of one or more of the following:
(a) a polypeptide comprising the amino acid sequence set forth in SEQ ID NO: 1;
(b) a polypeptide comprising an amino acid sequence having at least 70% (e.g., at least 95%, at least 96%, at least 97%, at least 98% or at least 99%) identity to the amino acid sequence set forth in SEQ ID NO: 1, but less than 100% sequence identity with the amino acid sequence set forth in SEQ ID NOs: 2 or 3, wherein said polypeptide is capable of modifying the C7-atom of deoxynivalenol (DON) and/or DON derivative selected from the group consisting of: 3-keto Deoxynivalenol, 3-epi Deoxynivalenol, 15-Acetyldeoxpivalenol, Nivalenol, Fusarenon-X (4-acetylnivalenol), 3-amino Deoxynivalenol;
(c) composition, kit, foodstuff, intermediate foodstuff; fodder, intermediate fodder; feed, intermediate feed; additive, intermediate additive; detoxifying agent, intermediate detoxifying agent, nutritional supplement, intermediate nutritional supplement, prebiotic, intermediate prebiotic or mixture/s thereof according to any one of the preceding claims;
for/in one or more of the following:
i) modifying the C7-atom of DON and/or DON derivative/s;
ii) producing a foodstuff, intermediate foodstuff; fodder, intermediate fodder; feed, intermediate feed; additive, intermediate additive; detoxifying agent, intermediate detoxifying agent; nutritional supplement, intermediate nutritional supplement; prebiotic, intermediate prebiotic and/or mixture/s thereof;
iii) degrading DON and/or said DON derivative/s and/or altering toxicity of DON and/or said DON derivative/s, preferably said one or more polypeptides are recombinant and/or isolated polypeptides; further preferably said altering toxicity comprising one or more of the following: detoxifying and/or intramolecularly rearranging DON and/or said DON derivative/s; most preferably said one or more polypeptides are capable of modifying the C7-atom of DON and/or said DON derivative/s;
iv) any combination of (i)-(iii);
v) use according to any one (i)-(iv), wherein said use is an in vitro, ex vivo or in vivo use.
